(19) United States
(12) Patent Application Publication
(10) Pub. No.: US 2017/0105707 A1
(43) Pub. Date: Apr. 20, 2017

(54) SAMPLE COLLECTOR

(71) Applicant: MEDDX SOLUTIONS LTD, Hereford and Worcester (GB)

(72) Inventors: Richard JOSEPH SENIOR, Hereford and Worcester (GB); Roderick Iain DAVIDSON, Hereford and Worcester (GB)

(21) Appl. No.: 15/276,388

(22) Filed: Sep. 26, 2016

Related U.S. Application Data
(63) Continuation of application No. PCT/GB2015/050898, filed on Mar. 25, 2015.

(30) Foreign Application Priority Data
Mar. 25, 2014 (GB) 1405297.1
Mar. 25, 2014 (GB) 1405298.9

(51) Int. Cl. A61B 10/00; B01L 3/00; G01N 33/52; A61B 5/15; G01N 21/78

(52) U.S. Cl. CPC A61B 10/007; A61B 5/150343; A61B 10/0038; G01N 21/78; G01N 33/521; B01L 3/505; A61B 2010/0061; G01N 2021/7759

(57) ABSTRACT

The present invention relates to a sample collector comprising: an inner flexible collection bag having an opening along its upper edge at least when open for use; and an outer support for supporting the collection bag at its opening. The support has sides that can fold together for storage and open to provide a structure in which one end has a spout and another end has a transverse portion. The sides include fold lines to enable bending of the sides from a storage configuration into an open configuration. The collection bag being connected to the outer support such that on opening of the outer support the bag is held open for collection of a sample.

The present invention relates to a sample collector, particularly though not exclusively for urine. The sample collector may include a preliminary testing kit.

Urine is routinely tested for a wide range of reasons, in particular drug testing associated with sports or addiction problems, infections such as STIs, cystitis, pregnancy, kidney stones or for raised sugar levels in diabetes. Samples of urine must be collected into a sterile container to avoid contamination and thus false results.

Men are easily able to direct their urine stream into a small pot, while women find this more difficult. As a result samples given by women are typically initially delivered into a large open container and transferred into a smaller sample container, which can then be sent to a laboratory for testing. The use of the additional container, in particular containers not specifically intended for urine collection, increases the risk of contamination. In addition the transferring of the sample from one container to another can cause spillage, which should also be avoided.

Cartons comprising a stiff outer wall, and a flexible plastics insert or liner are known. These are generally provided with four sides, which can collapse to a flat state for storage. Such containers can be used as food containers, or flower vases for example.

The object of the present invention is to provide an improved sample collector.

According to a first aspect of the invention there is provided a sample collector comprising: —

an inner flexible collection bag having an opening along its upper edge at least when open for use; and

an outer support for supporting the collection bag at its opening, the support having sides that can fold together for storage and open to provide a structure in which one end has a spout and another end has a transverse portion, the sides including fold lines to enable bending of the sides from a storage configuration into an open configuration,
the collection bag being connected to the outer support such that on opening of the outer support the bag is held open for collection of a sample.

According to a further aspect of the invention, there is provided a sample collector comprising:

an inner flexible collection bag having an opening along its upper edge at least when open for use; and

an outer support for supporting the collection bag at its opening, the support being formed with sides having fold lines configured to allow it to conform to:

a first shape in which the sides are substantially flat against one another; and

a second shape in which the outer support forms a prism-like configuration;

the collection bag being connected to the outer support such that on opening of the outer support the bag is held open for collection of a sample.

According to a further aspect of the invention, there is provided a sample collector comprising:

an inner flexible collection bag having an opening along its upper edge at least when open for use; and

an outer support for supporting the collection bag at its opening, the support having sides that can fold together between a storage configuration and an open configuration, the sides including fold lines to enable the outer support to be snapped from one position to the other and to remain stable and self-supporting in the open position;

the collection bag being connected to the outer support such that on opening of the outer support the bag is held open for collection of a sample.

According to a further aspect, the present invention provides a kit for collecting a sample, the kit comprising:
a sample collector as herein described; and
a sealable container for receiving a sample collected in the collector.

The kit may further comprise packaging for enclosing the sealable container. For example, a leak-proof package may be provided to enable the sealable container (and sample therein) to be returned for testing via mail.

Preferably the collection bag has a removable extension above its opening, forming a closure to the bag prior to use. The extension is joined to the bag by a tear line. For example the tear line may comprise a line of micro-perforations. Alternatively the micro-perforations may comprise laser scribes (which form a tear line without compromising the sealing of the bag to maintain its sterility). The tear line provides a removable, or tear-off, top for the collector bag. Before use the extension can be torn off along the micro-perforations, to provide the opening. This ensures that the inside of the bag is sterile before use.

Preferably, in the open position the outer support provides an opening for supporting the collector bag. For example, the outer support supports the upper end of the collection bag. The upper end of the collection bag may for example be fixed or bonded to the outer support (for example outer surfaces of the bag may be fixed to adjacent inner surfaces of the outer support). Preferably, in the open position the outer support forms a multifaceted structure. Preferably, in the open position the outer support forms a multifaceted structure providing an irregular shaped opening providing a spout.

Preferably in the open position the outer support forms a prism like configuration having a substantially triangular or wedge shaped cross-section. Alternatively the prism may be an irregular shape with a spout at one end, with the other end having a plurality of sides. In the storage position the support is essentially flat with one side flat against the other. Typically the sides are scored, perforated or pre-folded along the fold lines at a position such that on pushing an edge of the flat device closest to the lines, the device bends or folds, moving into the open position.

Preferably, the outer support comprises at least one pair of prefolds, perforations or scores. Preferably, the outer support comprises at least one pair of prefold, perforations or scores diverging away from each other, or converging towards each other, towards the opening. Preferably, the outer support comprises a first end and a second opposed end, in which the opening is provided by the second end. Preferably, the at least one pair of prefolds, perforations or scores diverge away from each other, or converge towards each other, in a direction extending from the first end towards the second end of the outer support.

The perforations, scores or pre-folds can be curved to ensure that the cylinder is self-supporting and will not collapse when held. This creates a transverse end having a concave shape. Optionally, for greater stability, the upper section of the perforations or scores can be provided with a second line of perforations curving away from the first, the join being between a half and a third of the height of the support. This provides a support having a three-sided bottom edge and a five-sided top edge.

Preferably the outer support is provided with at least one window cut out. Typically one window will be provided with a mark to indicate a minimum fill level. A maximum fill line may also be indicated. Typically another window will be provided with a temperature gauge to indicate the temperate of the urine. Alternatively the fill levels and the temperature gauge may be provided in the same window. Other windows may also be provided.

In one embodiment the collector can be provided with test sticks, such as are readily available, which can be inserted into the sample once taken. One or more test sticks can be provided adhered to the inside of the liner. A further window may be provided for reading of the results. Alternatively test sticks can be provided on an extension of the support such that they can fold into the sample once collected. In a further embodiment the sticks can be provided in a rack attached to the liner, so that they can extend out of the collector during collection and can be pushed into the sample, once collected. Where the test sticks are provided adhered to the inside of the bag, they will be sealed within the bag extension prior to use, ensuring that they are sterile.

The test sticks may for example be provided within a sachet which is attached to the outside of the support. The sachet may be opened and the test sticks dipped into the liquid after collection. The test sticks may for example be provided on hooks, tabs or slots within the collector bag. The test sticks may be arranged within the collector bag to be located at a certain height from the base of the collector bag such that the bag is required to be filled to a predetermined level in order for the test sticks to function. The first few milliliters of urine are not optimal for testing. To remove this from the sample collected, the inner flexible bag can be provided with a false bottom. Typically the false bottom will be created by a second seal above the lower seal of the liner, with the second seal having an opening the lowest point of the seal, to allow the urine to pass into the false bottom. While this may be anywhere along the seal, typically it will be distanced from the end with the spout. Usually is will be provided slightly towards the spout end of the portion forming the transverse portion when open. Thus in use the first urine to enter the liner will pass through the opening into the false bottom, and when full main flow of the urine will fill the rest of the liner.

In some embodiments the collector can be adapted for use with an evacuated test tube, such as for example a Vacuette® or a Vacutainer®. This is a container standardly used for collecting samples, typically blood. It is a sample container sealed by a rubber membrane seal and holding a vacuum prior to use. On piercing of the seal with a hypodermic needle, the vacuum draws fluid through the needle into the container. The outer support can be provided with an extension holding a hypodermic needle, connected via a tube to the liner. Importantly the extension can be adapted to be held closed for collection of the sample, to shield the user from the needle during sample collection. The extension may also be adapted to be arguable into an open position after collection, in which the needle is exposed, such that an evacuated test tube, such as for example a Vacuette® or a Vacutainer® may be connected to the needle for transfer of the sample.

To help understanding of the invention, six specific embodiments thereof will now be described by way of example and with reference to the accompanying drawings, in which:

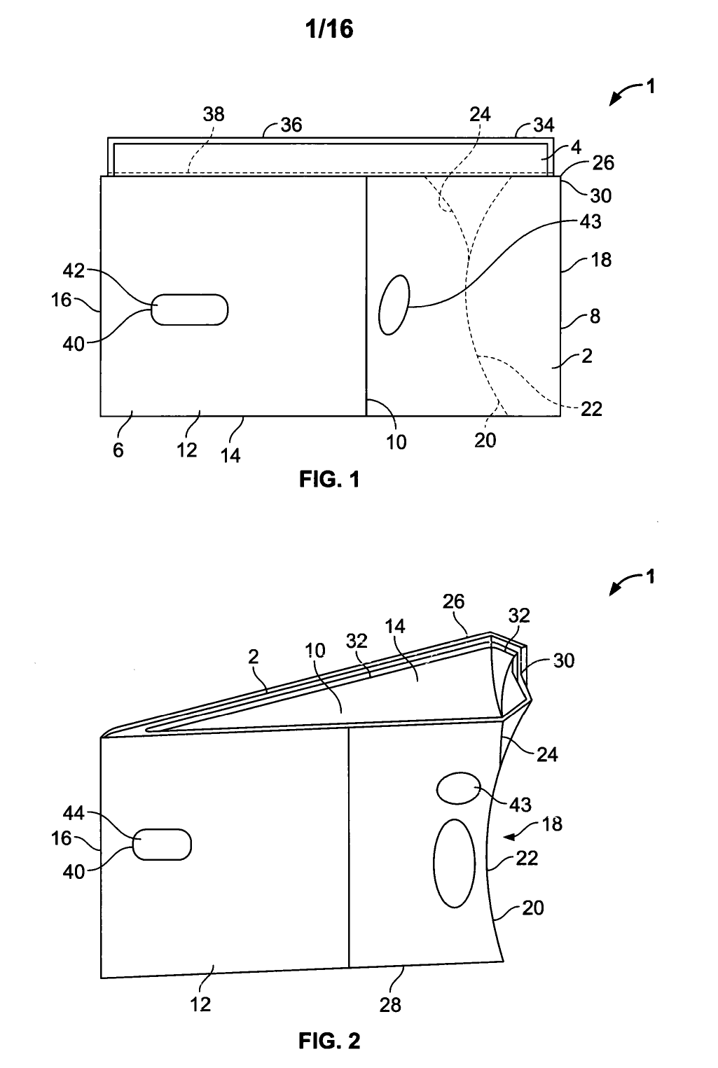

FIG. 1 is a front view of a sample collector in the closed position, according to a first embodiment of the invention;

FIG. 2 is a perspective view of the sample collector of FIG. 1 in the open position;

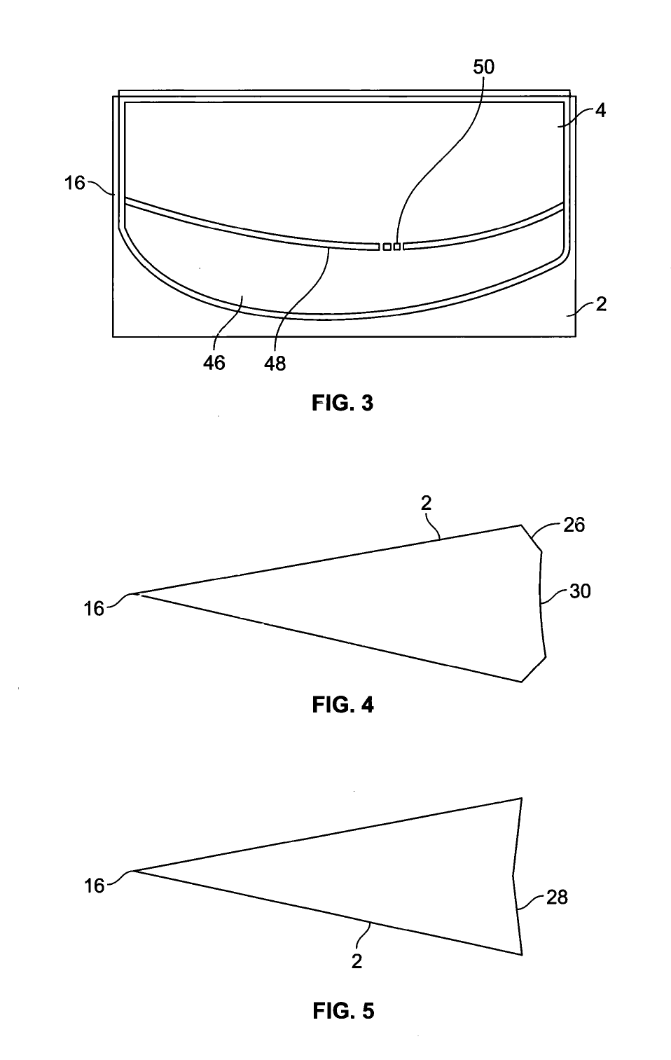

FIG. 3 is a cross-sectional view of the sample collector of FIG. 1, showing the liner;

FIG. 4 is a top view of the sample collector of FIG. 1;

FIG. 5 is a bottom view of the sample collector of FIG. 1;

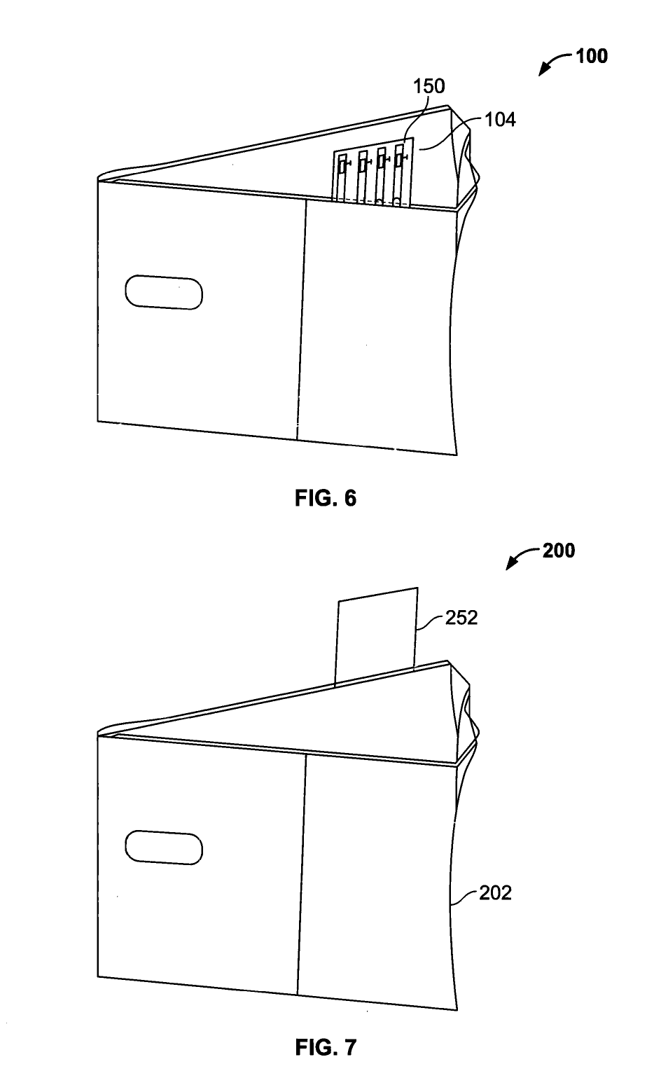

FIG. 6 is a top perspective view of a sample collector according to a second embodiment of the invention;

FIG. 7 is a top perspective view of a sample collector according to a third embodiment of the invention with the strip holding extension upwardly;

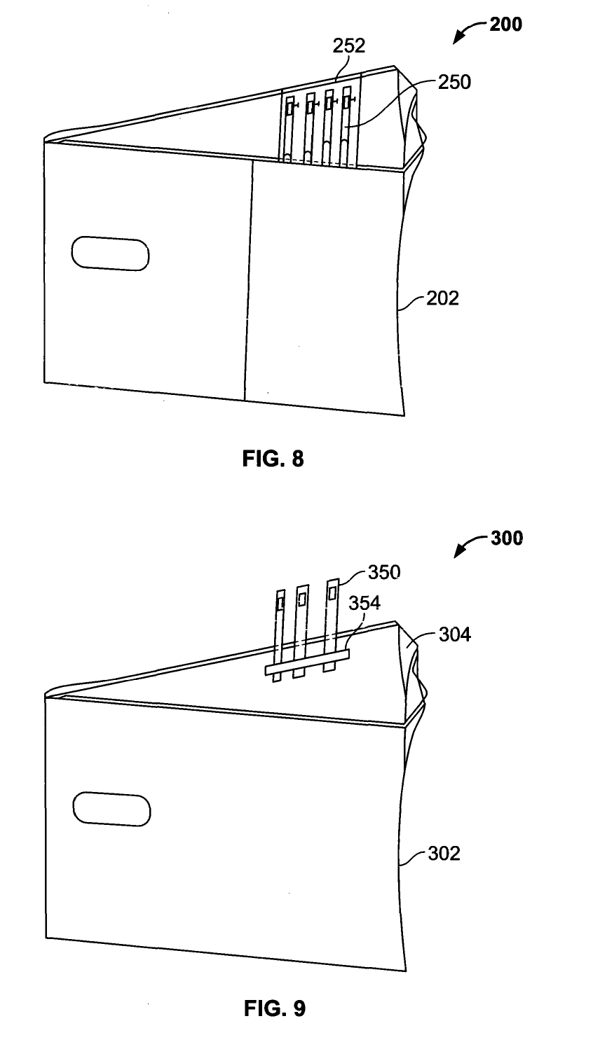

FIG. 8 is a top perspective view of the sample collector of FIG. 7 with the strip holding extension folded within the collector;

FIG. 9 is a top perspective view of the sample collector according to a fourth embodiment of the invention;

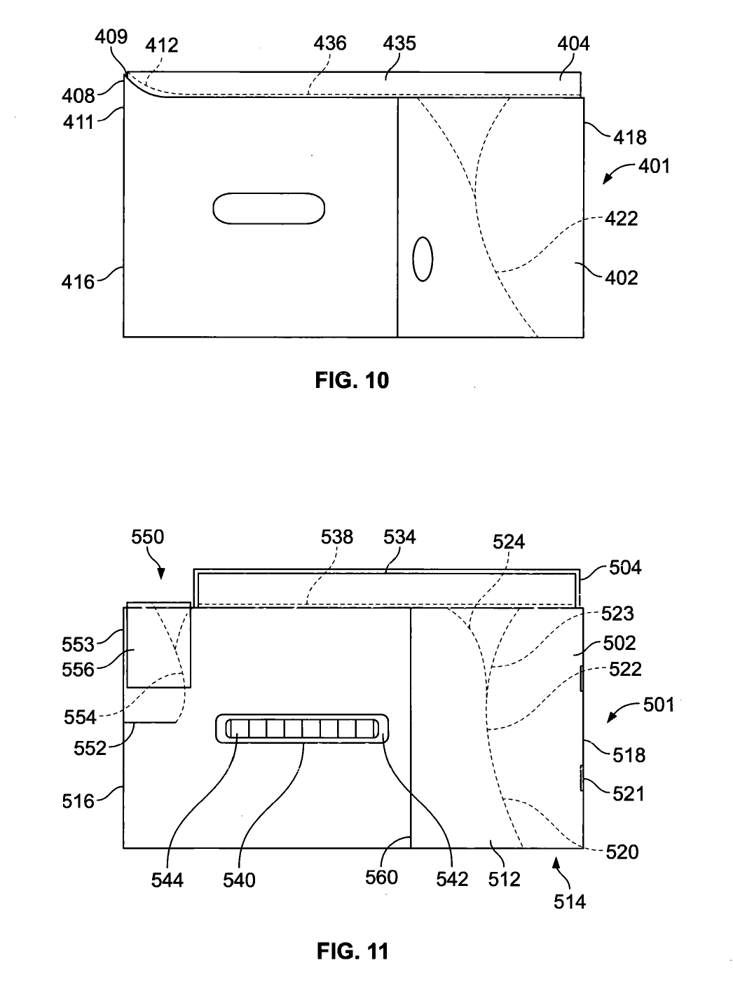

FIG. 10 is a front view of a sample collector according to a fifth embodiment of the invention;

FIG. 11 is a front view of a sample collector according to a sixth embodiment of the invention;

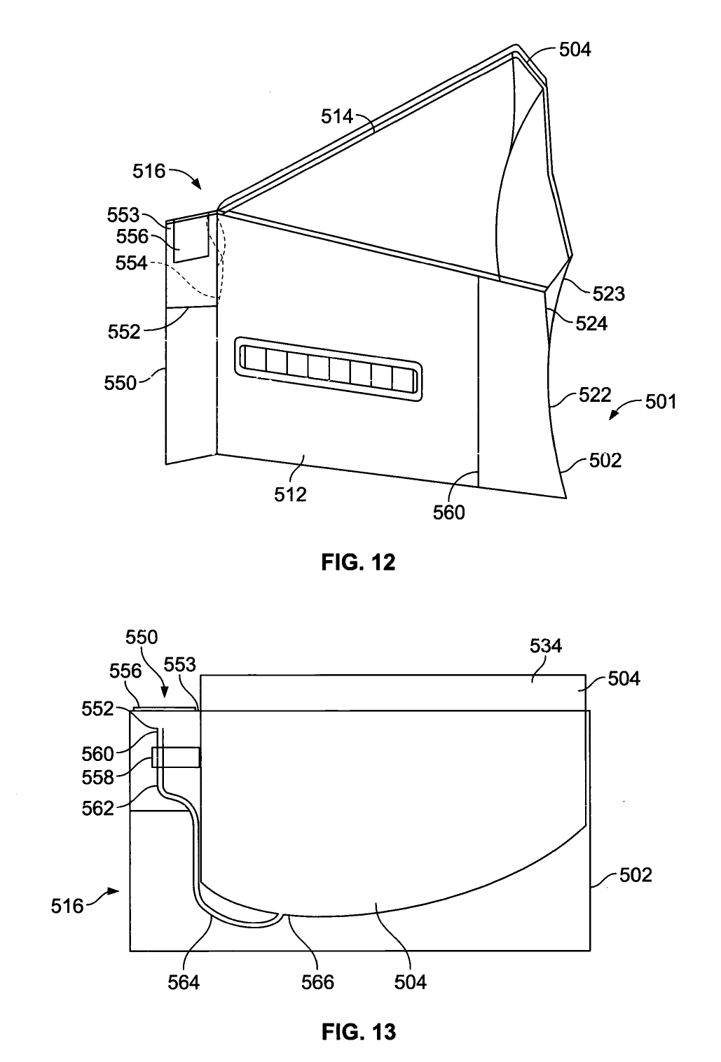

FIG. 12 is a top perspective view of the sample collector of FIG. 11;

FIG. 13 is a cut-away view of the sample collector of FIG. 1, showing the hypodermic needle and it connection to the liner;

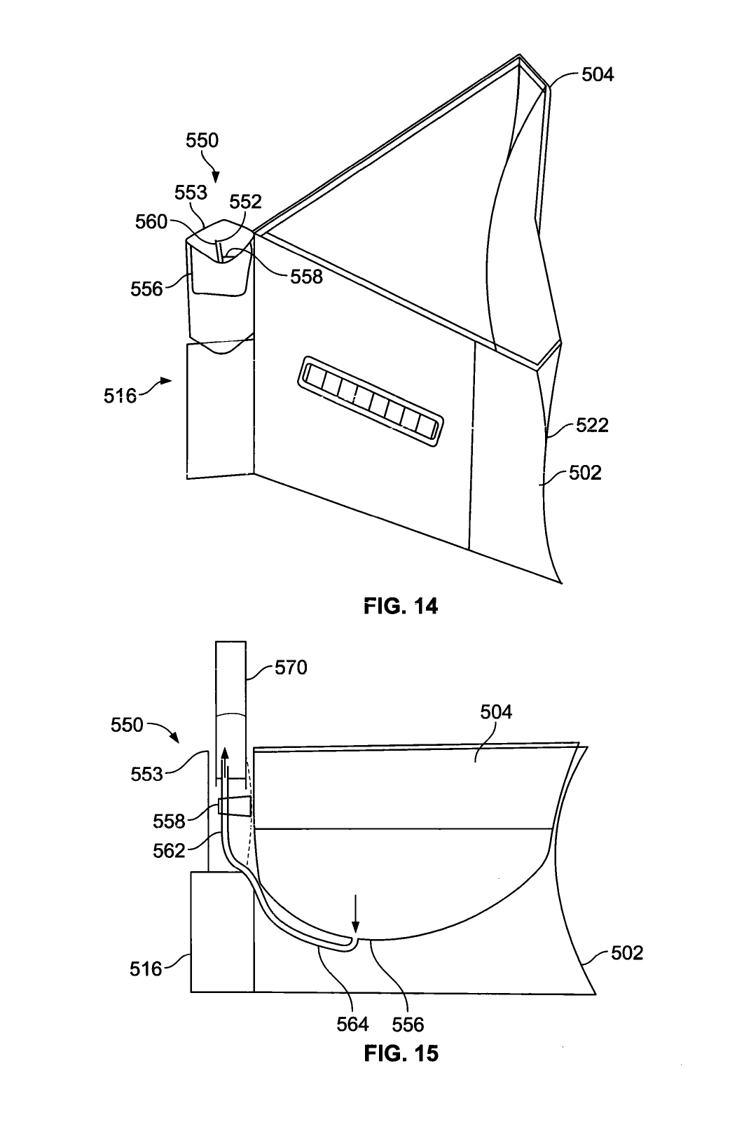

FIG. 14 a top perspective view of the collector of FIG. 11 in the fully open position;

FIG. 15 is a cross-sectional view of the sample collector of FIG. 11 including a Vacutainer®;

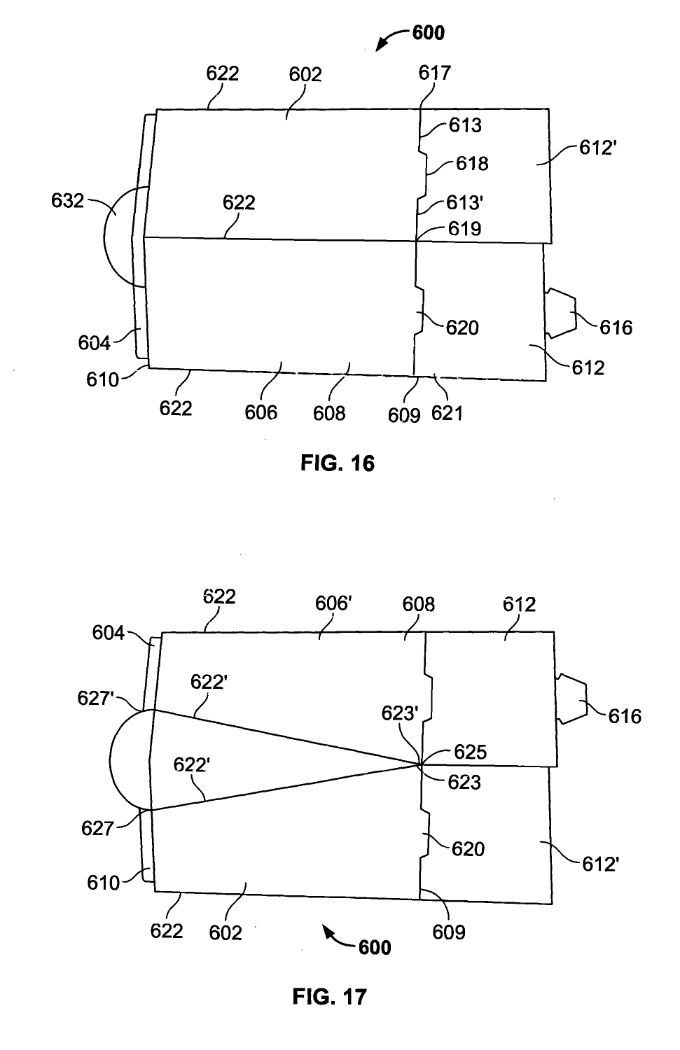

FIG. 16 is a perspective rear view of a collector according to a further embodiment of the present invention, showing the collector in the stored position;

FIG. 17 is a perspective front view of the collector of FIG. 16, showing the collector in the stored position;

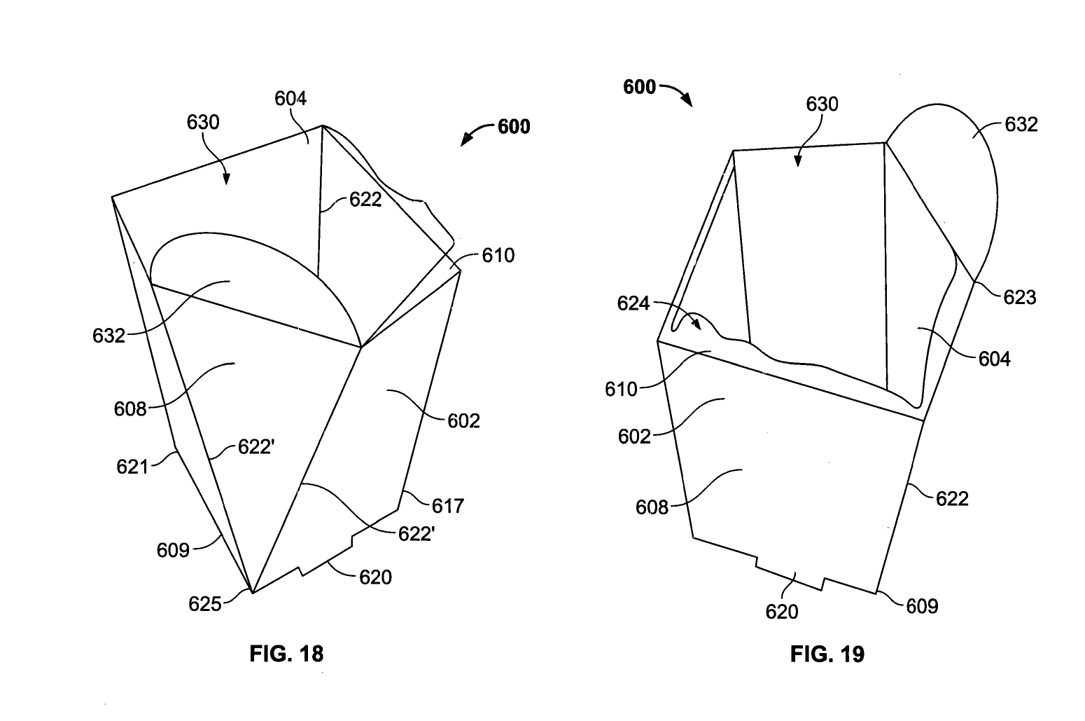

FIG. 18 is a perspective front view of the collector of FIG. 16, showing the collector in the open position;

FIG. 19 is a perspective rear view of the collector of FIG. 16, showing the collector in the open position;

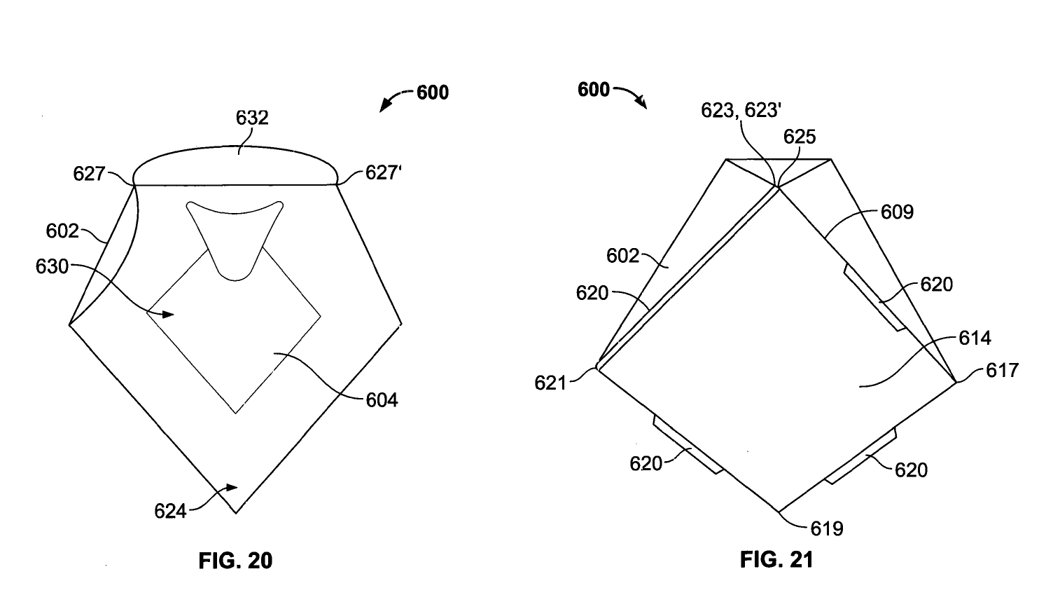

FIG. 20 is a view from above of the collector of FIG. 16 in the open position;

FIG. 21 is a view from below of the collector of FIG. 16 in the open position;

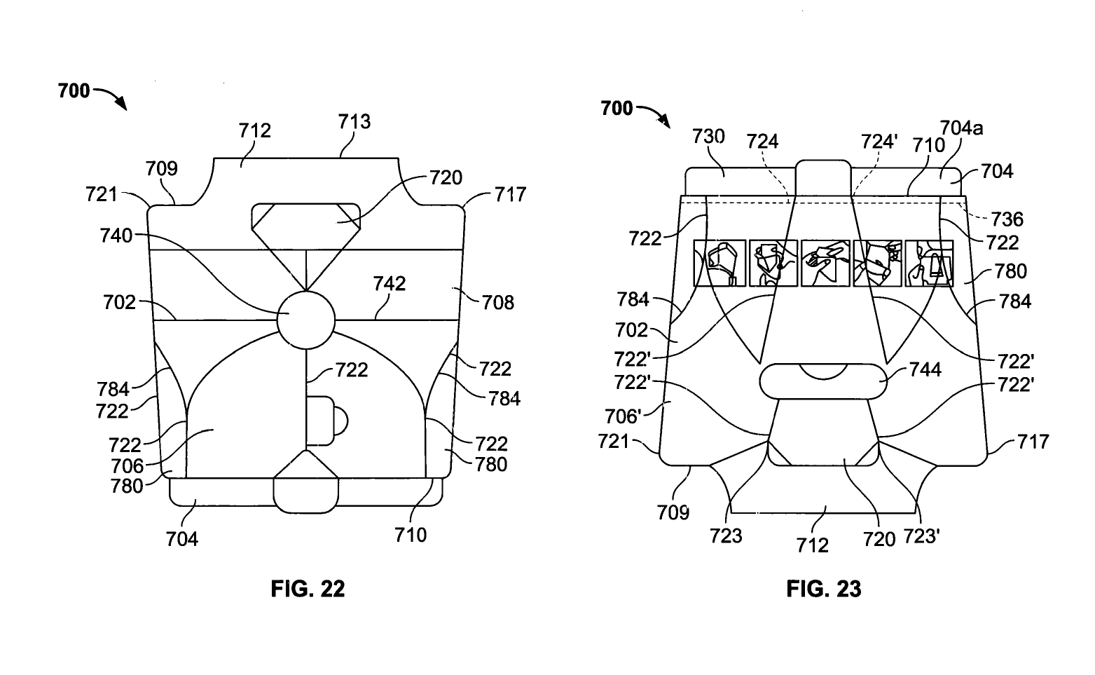

FIG. 22 is a view from the front of a collector according to a further embodiment of the present invention, showing the collector in the stored position;

FIG. 23 is a rear view of the collector of FIG. 22, showing the collector in the stored position;

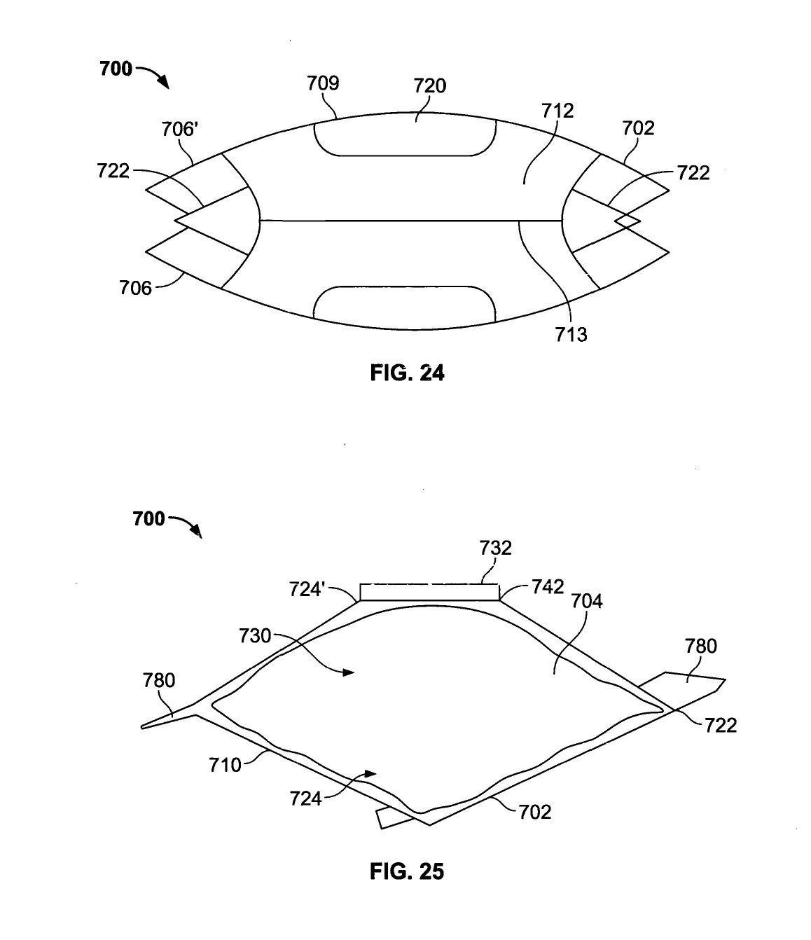

FIG. 24 is a view from above of the collector of FIG. 22, showing the collector in the open position;

FIG. 25 is a view from below of the collector of FIG. 22, showing the collector in the open position;

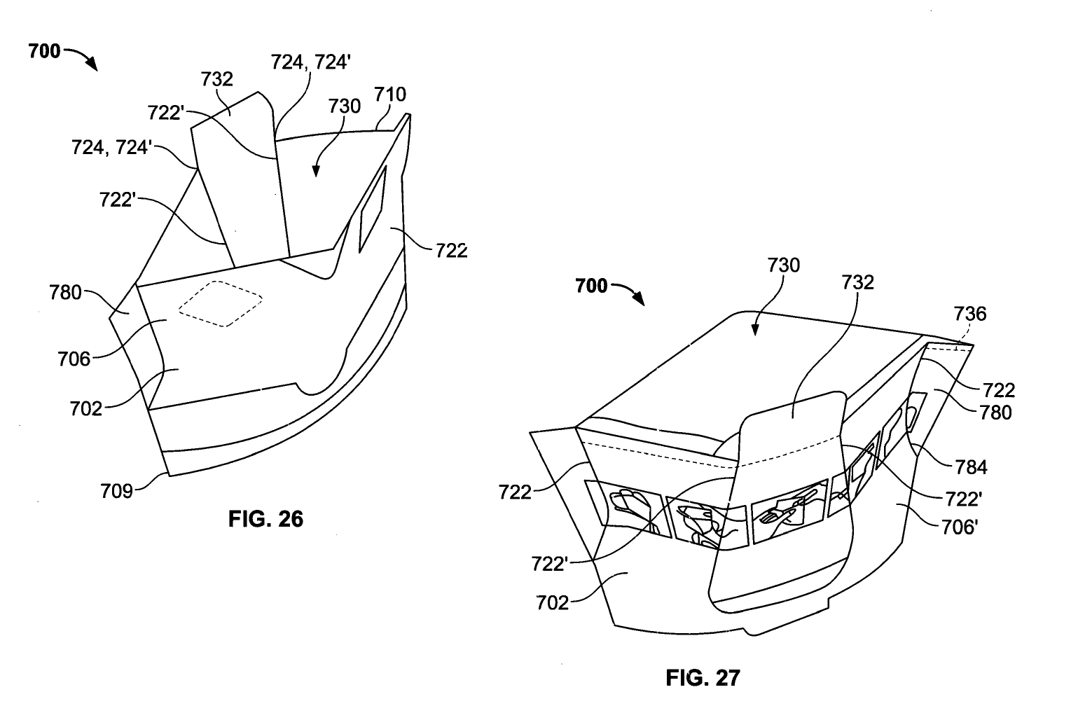

FIG. 26 is a perspective view of the collector of FIG. 22, showing the collector in the open position;

FIG. 27 is a perspective view of the collector of FIG. 22, showing the collector in the open position; and

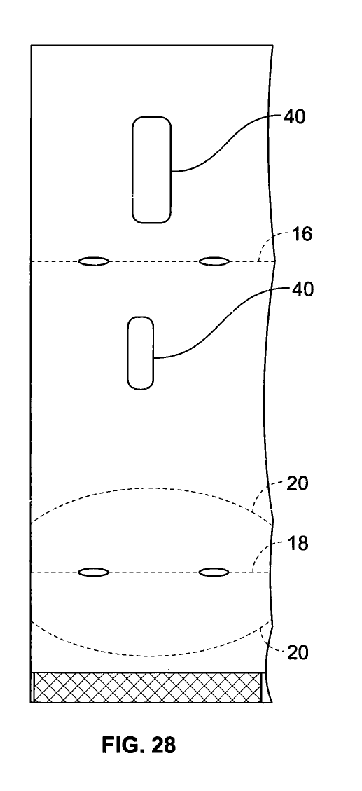

FIG. 28 is a net suitable for forming the outer support in of the embodiment of FIG. 1;

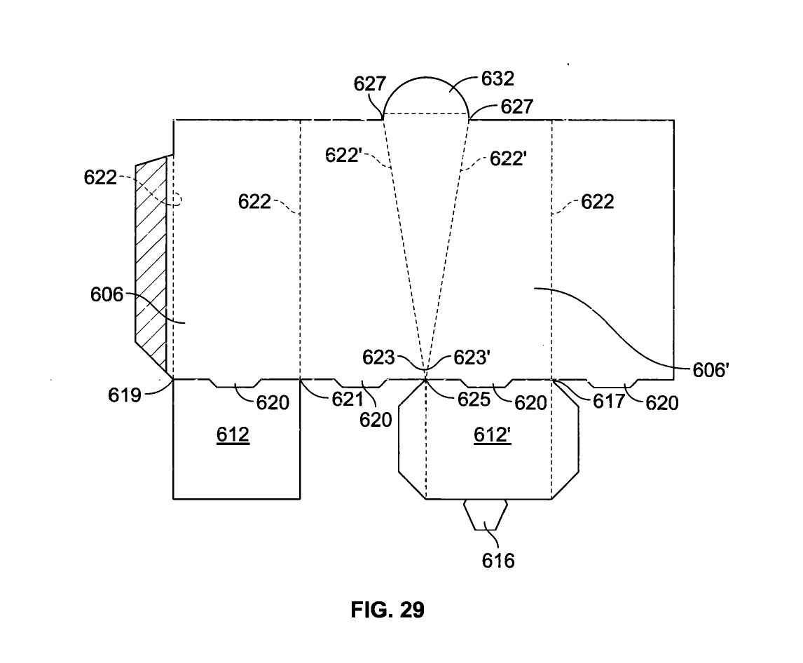

FIG. 29 is a net suitable for forming the outer support in of the embodiment of FIG. 16; and

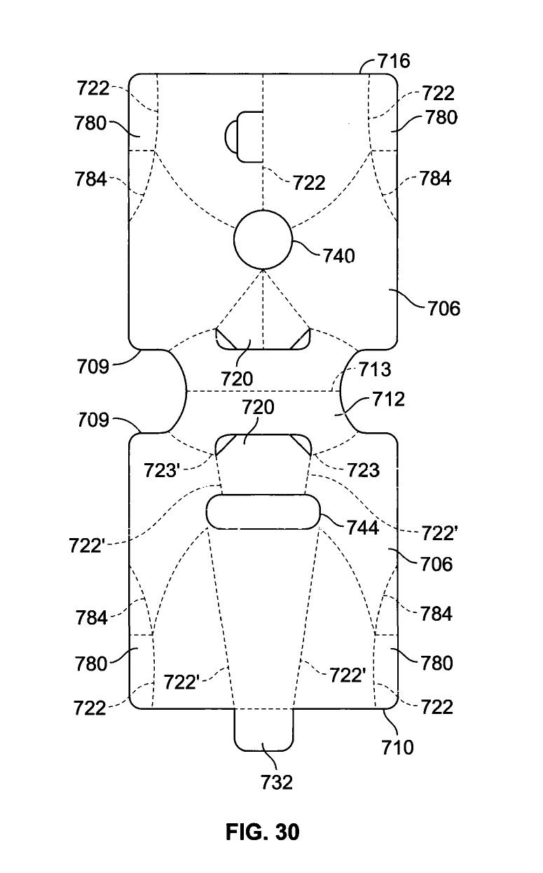

FIG. 30 is a net suitable for forming the outer support in of the embodiment of FIG. 22.

Referring to the Figures, the sample collector 1 comprises an outer support 2 and a flexible, waterproof inner lining or bag 4, for collecting a sample, typically urine.

The support 2 is formed from a sheet of material, typically card, although plastics material, such as polyethylene or polypropylene could also be used. The sheet may be a laminate paperboard with a fully coated exterior. The material could be a biodegradable or recyclable material. As shown the sheet has been folded around and adhered back on itself 10, however two or more sheets could be adhered together to form the support.

The outer support can conform to at least two shapes, one, as shown in FIG. 1, in which the sides 6 are substantially flat against each other, and a second, as shown in FIG. 2, in which the support forms a substantially three-sided prism-like device. The first conformation will be referred to as closed, and the second as open. The outer support is scored or folded 8, 20 such that it can lay flat with its two sides 12, 14 one against the other, or alternatively form an essentially triangular shape, as viewed from above. This shape is particularly convenient as it provides a pouring spout 8, which can easily be inserted between the legs of a person to collect urine.

As shown the support is provided with folds 8 at both ends 16, 18 enabling the support to lie flat for storage and transport. Between 25 and 35% of the length of the flat support, perforations 20 are provided, which indicate a bending position of the support. Alternatively the bending position can be indicated by scoring the support or pre-folding the support or a combination of all of these methods to create fold lines.

The score line 20 is provided as a curve to provide stability to the outer support in the open position. The curve is concave with respect to the end 16 of the spout in the open position. In this position the back edge 18 of the device in the closed position is urged into a position where its centre is further inwards of the triangle than its top or bottom. As a result it will return to its flat position only if pushed on from the inside. This allows the device to be snapped from one position to the other and remain stable and self-supporting in the open position.

For further stability the uppermost section of the perforated or scored line 22 may be divided into two with a second section 24 curving in the opposition direction from the main line. Referring to FIGS. 4 and 5, this provides a shape having three sides at one end, and five sides at the other. As shown the five sided end forms the top 26 with the three sided end forming the bottom 28, however the support could also be used the other way up. In a further alternative the both ends of the curve are provided with additional elements. These additional lines are not essential to the forming of the open shape, but do provide additional stability and make the device easier to hold.

In addition the end 18 of the support where the additional scores or perforations are provided is slit 30. This allows the end to separate or overlap, allowing the centre of the end to extend further inwards, again adding further stability to the support.

A waterproof liner 4 or bag is adhered just below the top inside edge 32 of the support, into which the sample can be collected. The liner will be made of polypropylene, polyethylene, or any other suitable film or membrane. The liner may be formed from a polymer with heat sealed seams. Typically, a gusset may be formed in at least a lower portion of the liner to allow the liner to be expanded to the open position in use. Alternatively the liner can also make of a biodegradable or recyclable material, which is waterproof for the time of use of the device. Providing the liner from a flexible material enables it to follow the contours of the support, and lie essentially flat within the liner for storage and transport, and substantially conform to the shape of the support when open. The liner is generally adhered to the top of the support only, but may also be adhered at various other positions, particular where windows or other elements are provided (see later).

To ensure that the liner 4 is free from contamination before use, an extension 34 to the liner is provided extending above the top of the support. Prior to use the liner is sealed along all of its edges 36. Micro-perforations 38 are provided substantially level with the top of the support, to provide a tear guide. Thus on use, the section of liner above the micro-perforations can be torn off, the micro-perforations making tearing easy, and the support is snapped into its open position for use. This ensures that the inside of the liner is sterile before use.

As shown the support is provided with cut-outs 40 on either side, providing windows to the liner. At a position level with the cut-out 40 on one side, the liner will be marked with fill levels 42, as shown a minimum and a maximum, although a minimum fill level alone may be provided. These give an indication to the user of the level to which she should attempt to fill the sample collector.

In addition, the sides of the support 2 can be provided with a textured surface, for example pimples or dimples, either along the whole outer surface, or in discrete areas, 43, to provide finger points to assist in grip of the device by a user, and as an indication of where the device can be held.

One known method of evading drug testing is to provide another person's urine, or an earlier sample of one's own urine, for testing. To ensure than the sample collected is fresh and not one collected earlier, the collection device is also provided with a temperature gauge or thermometer 44. This is adhered to the liner at a position corresponding to a window in the support 2. The temperature gauge may also be adhered to the support, at a position where it will contact the liner. This may include adhering the liner to the support at an adjacent position.

Referring to FIG. 3, when testing, the first few millilitres of urine does not include the same level of solutes as the main portion of the urine. As such the first few millilitres, typically 30 ml, should be discarded and not tested. In order to prevent the first few millilitres of urine for mixing with the rest of the urine collected, the liner 4 can be provided with a false bottom 46, which can be used to collect and hold the this part of the sample. To create the false bottom 46, an additional seal 48 is provided extending most of the way along the length of the lining. An opening 50, or perforated area, is provided at a low point in this second seal, 48. This will be at a distance from the spout end. Generally the opening will be at a position slightly to the spout end of the fold line 22. This is because the bag 4 will fold, crimple and wrinkle behind the fold line 22 in the open position. As a result the initial portion of the urine sample passes into the liner, through the opening 50, and into the lower area 46. Once this is filled, the rest of the sample is held above the upper seal, with very little movement of fluid across the opening. The lower area, below the false bottom, will typically hold 10-40 ml of fluid. On tipping of the sample into a container for sending to a laboratory for testing, the fluid in the false bottom will remain there, with only the main flow sample being transferred.

The sample collector 1 may also be used to test urine straight away, before the urine is sent off to a laboratory. The device can be provided with sample testing sticks, such as are commercially available. These are strips of plastics material onto which a coating has been adhered, which reacts in some way on contact with a particular substance or solute. Typically the coating will change colour, or will cause a line to appear, to indicate a positive result. One common example is pregnancy-testing sticks. However, similar testing sticks are available for testing blood sugar levels, for diabetics, or for the presence of particular drugs such as cannabis, opiates, or other substances banned by sports regulation bodies. In further examples test strips are know to test for the presence of infections, in particular STIs, or for kidney stones.

The test strips may be provided with the sample collector, for example in a separate packet. However, in accordance with an important feature of the invention, the test strips may be incorporated into the collector.

Referring to FIG. 6, the sample collector there shown 100 is provided with an array of test strips 150 adhered to the inside of the liner 104 such that once urine is collected in the liner, the strips are partially immersed in the urine and the test will be carried out.

As shown in FIGS. 7 and 8, the sample collector 200 has an outer support 202 having a fold down extension 252 and with test strips 250 provided on this extension. The extension will extend substantially vertically (in use) above the support during collection of the urine, as shown in FIG. 7. Once the sample has been collected and the temperature verified, the section of the support carrying the strips can be folded down, as shown in FIG. 8, placing the strips into the sample. Alternatively, the extension 252 may be folded down over the outside of the support 202 during collection of the sample (not shown), and then after verification of the temperature, folded over into the inside of the collector, where the strips will contact the urine and testing carried out.

A further alternative is show in FIG. 9 the sample collector 300 is provided with a rack 354 adhered to the inside of the liner 304. The rack 354 holds one or more strips 350. Before sample collection the strips will extend upwards out of the rack. However, once the sample has been collected and the temperature verified, the strips can be pushed through the rack to extend into the sample, testing the urine.

Sample collectors can be made with the appropriate testing strip or strips. For example single testing strips can be provided to testing for pregnancy or sugar levels, or a selection of strips can be provided for drug testing.

In the embodiments shown in FIGS. 6 and 9, the extension 34 of the liner, as described in relation to the embodiment of FIG. 1 can be sufficiently long to cover and seal the strips. With respect to the embodiment of FIGS. 7 and 8, the sample collector can be provided within a sealed pouch (not shown) to ensure that the test strips, and the sample collector as a whole, are sterile before use. Where a pouch is provided the extension 34 may be omitted.

After testing, or in situations where no test is carried out straight away, a sample of the urine will be send to a laboratory for further testing. The spout 16 enables ease of pouring of the urine into a laboratory sample tube, which can then be sent to the laboratory for full testing.

Referring now to the embodiment shown in FIG. 10, the sample thereshown is very similar to that shown in FIG. 1, with the exception that the spout 408 is provided with an extended upwards facing tip 409, to provide for ease of pouring of the collected sample from the collector 401. The sample collector 401 comprises an outer support 402 and a waterproof lining 404. As before, the sample collector is able to adopt two positions: a flat position, as shown, and an open position for use. The outer support 402 is scored 422 at fold lines to enable it to adopt the substantially triangular prism shaped open confirmation. In this open confirmation the outer support 402 has a spout end 416 and a transverse end 418. The liner 404 is adhered to the inside of the support 402 and is provided with a tear-off strip 435, with micro-perforations of a laser formed tear line. 436. The line 436 follows the top 440 of the outer support 402. Once opened, the flexibility of the liner 402 allows it to adopt the confirmation of the support 402.

The top 440 of the outer support 402, at the spout end, is provided with an up-turned tip 409. In the open position this provides a lip 411 for the spout to ease pouring from the sample collector. The tear line 436 follows the line of the top 440 of the outer support, and this creates a lip 412 on the liner 404 as well. This ensures that a user can pour the collected sample, or a portion of the collected sample, into another container, for transport to a laboratory for testing.

Now turning to FIGS. 11 to 15, the embodiment thereshown is designed for use in conjunction with a Vacutainer® produced by Becton, Dickinson and Company of the USA. A Vacutainer® is a container which holds a vacuum before use and is sealed with a rubber membrane. The membrane can be pierced by a hypodermic needle, with the vacuum then drawing fluid through the needle into the container. The membrane is self-sealing.

The sample collector 501 comprises an outer support 502 and a flexible, waterproof inner lining 504, for holding a sample, typically urine.

The outer support 502 is made from a single piece of material of substantially rectangular shape, which has been folded back on itself, with its ends joined and then pressed flat, creating two sides 512, 514. As in the first embodiment it is usually made from a paper material, generally card, but it could be made of plastics materials. The material can be biodegradable or recyclable

The outer support is also adapted to adopt an open position as shown in FIG. 12. In the open configuration the sample collector forms a triangular prism shape, having one end 516 pointed, and the other end 518 wide.

The outer support 502 is scored on both sides approximately 25-35% from the end 518 that will be wide in the open configuration. The scoring 520, which may also include perforations, or larger apertures, indicates fold lines to enable the collector 501 to adopt the open position.

To provide stability to the open position, the fold line 520 is provided as a curve 522, curving towards to the pointed end 516 of the shape when open. In this position the wide end 518 of the device is curved into the body of the collector, in an over-centre manner. Thus giving the shape stability. Once urged into this position, the collector will return to its closed position only in the event of pressure applied to the inside of the device. This curving allows the device to be urged from one position to the other, and remain stable and self-supporting in either position.

The top of the score or perforation line 522 may be divided with one section 523 following the line 522, while the other section 524 curves in the opposite direction. This provides additional structure to the open confirmation, creating a shape that has three-sided cross-section at the bottom and a five sided cross-section at the top. However, the additional curves could be position at the bottom as an alternative or an addition.

The liner 504 is waterproof to contain the urine or other sample collected in the collector. Usually the liner will be made of polyethylene or polypropylene, but any other film or membrane capable of holding the sample may be used. The liner can alternatively be made of a biodegradable or recyclable material, which is waterproof and capable of holding the sample for the life-time of the sample collector in use. The liner is made of a flexible material to enable it to conform to the shape of the outer support 504 in both of its confirmations. The liner is adhered to the outer support 502 just below its top edge 532, but may be adhered at other positions as well.

To ensure that the liner is free from contamination before use, an extension 534 to the liner is provided above the height of the outer support. Prior to use the liner is sealed along all of its edges, including the top of the extension 534. Micro-perforations 538, or a laser tear line are provided at substantially the level of the outer support.

Again as in the first embodiment a minimum fill level will usually be indicated, 542 and possibly a maximum fill level, provided at or adjacent an window 540 in the outer support 502. In addition a temperature gauge or thermometer 544 will usually be provided. This enables a technician to check that the sample provided is substantially at body temperature and thus is fresh. The temperature gauge or thermometer is adhered to the liner at a window 540 in the outer support, thus providing good thermal contact with the lining holding the sample while being visible to the user.

In this embodiment the outer support 502 includes an extension 550 at its pointed end 516. The liner 504 does not extend into this extension. Within this extension is provided a hypodermic needle 560. It is positioned with its tip 552 slightly below the top 553 of the extension. The needle is connected to a polyethylene flag 558, which is sealed to the outer support 502 and/or to the liner 504. The lower end 562 of the needle is connected to a tube 564, which in turn is connected 566 to the liner 504, in communication with the collected sample. Thus there is a direct passage for flow of collected sample from the base of the liner, through the tube 564 to the needle 560. The extension is held closed by a tape 556, protecting a user from the needle while the sample is being collected.

Once the sample has been collected the collector 501 is handed to a technician who checks the temperature of the sample. He then removes the tape 556 from the extension, exposing the needle.

The end of the outer support is also scored, and/or perforated 554 in a manner similar to the other end of the collector. On opening of the tape, extension 550 can also be opened, in a similar manner to opening the sample collector to collect the sample, exposing the needle 560, by pressing the extension and urging it apart, as shown in FIG. 14. The scoring 554 on the extension is similar to that on the main body of the sample collector, namely a curved line.

The opening of the extension 550 exposes the needle, which is positioned so that a Vacutainer® 570 can then be pushed onto the needle. The vacuum in the Vacutainer® draws up the sample from the collector, via the tube, as shown in FIG. 15. This provides a reliable and sterile way of transferring the collected sample into a container for transporting to a laboratory for testing.

As shown the needle 560 is connected via the tube 564 to the liner 504 at its base. In this arrangement, the sample transferred to the Vacutainer® would be at the bottom of the sample. However, the tube 564 may be connected to any part of the liner below the minimum fill line. A false bottom may be provided in the liner, as described with reference to the embodiment of FIG. 3. As before, the false bottom will contain the initial portion of the sample, which is not optimal testing. Where a false bottom is provided the tube 564 will be connected to the liner 504 above the false bottom to ensure that only the main flow portion of the sample is drawn into the Vacutainer®.

On removal of the Vacutainer® from the sample collector 501, the Vacutainer® will contain a sample for testing and can be sent to the laboratory.

With reference to FIGS. 16 to 21 and FIG. 29, the sample collector 600 comprises an outer support 602 and a flexible, waterproof inner lining or collector bag 604 located within the outer support 602 for collecting a sample, typically urine.

The outer support 602 is moveable between a stored position, as shown in FIGS. 16 and 17, and an open position as shown in FIGS. 18 to 21. The outer support 602 is provided with prefolds 622, 622′ enabling the outer support 602 to lie flat in the stored position for storage and transport.

As shown in FIGS. 16 and 17, in the stored position, the sides 606 and 606′ of the outer support 602 are substantially flat and lie against each other. In the stored position, the outer support 602 is substantially rectangular in shape.

The outer support 602 comprises a body 608 comprising a first end 609 and a second opposed end 610. Two substantially square flaps 612, 612′ are located at the first end 609 of the outer support 602. Prefolds 623, 623′ enable the flaps 612, 612′ to be foldable to provide, in the open position, a base 614 having a substantially square cross-section at the first end 609 of the outer support 602.

Each flap 612, 612′ comprises an engagement feature 616, 618 for mutual engagement, preferably releasable engagement, with an engagement feature 618, 616 provided by the other flap 112′, 112. In the illustrated embodiment in FIGS. 16 to 21, flap 612 comprises a projection 616 shaped, dimensioned and positioned to be received within and to releasably engage a corresponding opening 618 provided by flap 612′ when the collector 600 is in the open position.

It is to be understood that the flaps 612, 612′ may comprise any number and combination of engagement features for mutual engagement, preferably releasable engagement.

Although the illustrated embodiment comprises a base 614 having a substantially square cross-sectional shape, it is to be understood that the base 614 may have any suitable cross-sectional shape, such as for example but not limited to circular, oval, triangular, rectangular, pentagonal or hexagonal, depending on the particular requirements for the collector.

As shown in FIG. 21, the first end 609 and the base 614 further provide four spaced apart protrusions 620 extending substantially perpendicular to the plane defined by the base 114 when in the collector 600 is in the open position. The protrusions 620 extend substantially parallel to the adjacent side 606, 606′ of the support 602. The protrusions 620 are shaped, dimensioned and arranged to support and/or stabilise the sample collector 600 when placed upon a support surface. Although the illustrated embodiment has four spaced apart protrusions 620, it is to be understood that the base 614 and the first end 609 of the support 602 can provide any suitable number of projections 620.

As shown best in FIGS. 16 and 17, the outer support 602 comprises a plurality of prefolds 622, 622′ extending from the first end 609 to the second end 610 of the support 602. The outer support 602 comprises three first prefolds 622. It may be noted from FIG. 29 that one of the prefolds 622 is formed at an edge of the outer support 602 which includes a tab (for receiving adhesive in order to assemble the support 602 during manufacturing). Each first prefold extends from a separate corner 617, 619, 621 of the base 614 at the first end 609 of the support 602. Each first prefold 622 extends substantially perpendicular to the plane defined by the base 614 in the open position. Each first prefold 622 extends substantially parallel to the other first prefolds 622.

It will be appreciated that it is not essential that the base 614 of the support 602 is closed (since it may only be closed by the liner when the support 602 is in the open position). As such, it will be appreciated that the plane defined by the base as referred to above may be the plane which extends between the opposed side portions at the end 609 of the support 602.

As shown best in FIG. 17, the outer support 602 further comprises a pair of diverging second prefolds 622′. The second prefolds 622′ each having a first end 623, 623′ located at the same corner 625 of the base 614. The second prefolds 622′ extend away from each other towards the second end 600 of the support 602. Each second prefolds 622′ extends at an non-perpendicular angle to the plane defined by base 614, or the plane defined between opposing side portions of the support 602. The non-perpendicular angles subtended by the second prefolds 622′ are preferably equal to each other. It is however to be understood that the second prefolds 622′ may extend at any suitable angle with respect to the plane defined by the base 614. The second ends 627, 627′ of the second prefolds 622′ are preferably located equally spaced from the adjacent first prefolds 622 at the second end 610 of the outer support 602.

With reference to FIGS. 18 to 20, in the open position the second, upper, end 610 of the support 602 provides an opening 630 having a substantially pentagonal cross-section. It will be noted that the diverging prefolds 622′ provide an arrangement in which the second upper end 610 has an additional side in comparison to the square base end 609. Thus, the effect of the perpendicular 622 and non-perpendicular prefolds 622′ is to provide an upper profile which includes provides a spout 624 and an opposing transverse portion (defined between the prefolds 622′). A lip 632 extends from the second end 610 of the support 602. The lip 632 extends between the second ends 627, 627′ of the pair of second prefolds 622′. The lip 632 provides additional stability in the open position and may either be used as a handle or could be folded inwardly into the open end 610 of the support to further secure the open arrangement.

In use, the user pushes a pair of prefolds 622 at the edges of sides 606, 606′ of the support 602 inwardly towards each other to move the support 602 from the flat, storage position to the open position. The flexibility of the collector bag 604 allows the bag 604 to adopt the configuration of the support 602.

With reference to FIG. 22, the collector 700 comprises an outer support 702 and a collector bag 704 located within the outer support 702 for collecting a sample, typically urine.

The outer support 702 is moveable between a stored position, as shown in FIGS. 22 and 23, and an open position as shown in FIGS. 24 to 27. The outer support 702 is provided with prefolds 722, 722′ enabling the outer support 702 to lie flat in the stored position for storage and transport.

As shown in FIGS. 22 and 23, in the stored position, the sides 706, 706′ of the outer support 702 are substantially flat and lie against each other. In the stored position, the outer support 702 is substantially rectangular in shape.

The outer support 702 comprises a body 708 comprising a first end 709 and a second opposed end 710. A flap 712 is located at the first end 709 of the outer support 702 and extends between opposed sides 706, 706′ of the outer support 702 to provide a base 714 in the open position (as shown in FIG. 24). The flap 712 comprises a prefold 713 enabling the flap 712 to lie flat in the stored position.

As shown in FIGS. 22 and 23, the first end 709 of the outer support 702 provides two protrusions or legs 720 located on opposed sides 706, 706′ of the outer support 702. The two protrusions or legs 720 extend substantially perpendicular to the plane defined by base 714 when in the open position. The protrusions 720 are shaped, dimensioned and arranged to support and/or stabilise the sample collector 700 when placed upon a support surface. Although the illustrated embodiment has two spaced apart protrusions 720, it is to be understood that the base 714 and/or the first end 709 of the support 702 can provide any suitable number of projections 220 in any suitable location or configuration.

The outer support 702 further comprises five prefolds 722 extend to the second, upper, end 710 of the support 702. The outer support 702 comprises two edges extending from a separate corner 717, 721 of the base 714 at the first end 709 of the support 702. Two of the prefolds 722 on each side of the support 702 are provided proximal to, but spaced apart from, the edges near to the second, upper, end 710. These folds define side wing portions on each side 702, 702′ of the outer support 702. It will be noted that in the open position (of FIGS. 24 to 27) the wing portions 740 of the two sides 702, 702′ remain together, and it will be appreciated that they provide a convenient means for bond (or otherwise attaching) the opposing sides 702, 702′ together.

Each first prefold 722 extends substantially perpendicular to the plane defined by the base 714 in the open position. Each first prefold 722 extends substantially parallel to the other first prefolds 722. The prefolds 722 adjacent to the wings 740 extend downwardly from the second, upper, end 710 of the support 702 into curved prefolds 784 (to assist in forming the open shape of the support).

The outer support 702 further comprises a pair of converging second prefolds 722′ extending in a direction from the first end 709 to the second end 710 of the support 702. Each second prefold 722′ has a first end 723, 223′ located at the first end 709 of the support 702 and spaced apart from each other. The second prefolds 722′ extend at a non-perpendicular angle to the plane defined by base 714. In the preferred embodiment the prefolds 722′ have an interrupted profile due to the cut out 744 (but this may not be the case in all embodiments). The non-perpendicular angles subtended by the second prefolds 722′ are preferably substantially equal to each other. It is however to be understood that the second prefolds 722′ may extend at any suitable angle from the plane defined by the base 714. The second prefolds 722′ extend inwardly towards each other from the first end 709 towards the second end 710 of the support 702. The second ends 724, 724′ of the second prefolds 722′ are spaced apart from each other at the second end 710 of the support 702.

As best shown in FIGS. 22 and 23, the collector 700 further comprises an extension 730 to the collector bag 704 provided above the height of the outer support 702. Prior to use the collector bag 704 is sealed along all of its edges, including the free end of the extension 730. Micro-perforations or a laser tear line are provided at substantially the level of the outer support. The collector 700 therefore comprises a tear off strip 736 following the line of the top of the outer support 702.

The outer support 702 further comprises a first window 740 enabling the user to view the collector bag 704. The outer support 702 further provides a minimum fill level indicator 742 located on a first side 706 of the support 702 aligned with the first window 740 (FIG. 20). A second window 744 is provided on the opposed side 706′ of the support 702 (FIG. 21). The second window 744 may be provided with a temperature gauge to enable a technician to check that the sample is substantially at body temperature and fresh. The temperature gauge may for example be adhered to the liner at the second window 744 on the outer support 702.

In use, the user first removes the tear off strip 736 from the collector 702 by tearing. User then pushes the prefolds 722 at the edges of sides 706, 706′ of the support 702 inwardly to move the support 702 from the flat, storage position to the open position. The flexibility of the collector bag 704 allows the bag 704 to adopt the configuration of the support 702.

With reference to FIGS. 24 to 27, in the open position the second end 710 of the support 702 provides an opening 730 having a substantially pentagonal cross-section. It will be noted that in the open position the section of the side wall 706′ between the pre-folds 722′ forms a transverse section (for example extending across the otherwise parallelogram form of the open end 710). Thus, the shape of the opening 730 defines a spout 724 (which is opposite to the transverse section). As in the previous embodiment a lip 732 extends outwardly from the second end 100 of the support 102. The lip 732 extends between the second ends 724, 724′ of the second prefolds 722′.

In the open position, it will be noted that the first, base, end 709 has a generally elliptical profile. The transition between the elliptical shape of the first end 709 and the pentagonal shape of the second end is enabled by the converging prefolds 722′ and the wings 780 extending only partially down the edges of the sides 706, 706′. In particular, it may be noted that the edges are not connected in a lower section so as to allow the edges to form divergent slits in the open position (which may provide increased stability).

The collector bag 704 collects the sample. The user can use the minimum fill level mark 742 to assess if sufficient volume of sample has been placed within the bag. The user may then decant/pour the sample collected into a sealable container (such as a conventional tube) which may have only a limited size opening.

For completeness, nets for forming outer supports suitable for use in the embodiments are shown in FIGS. 28 to 30. It will be appreciated that these nets would be preformed and glued into a support structure and provided with a liner in during a manufacturing process and then generally supplied in the closed configurations described above. As such, the collector provided to the end user would be ready for erecting from the closed position of the embodiments described above.

Although embodiments of the present invention have been described with reference to prefolds, it is to be understood that this term is used herein to broadly refer to a predetermined line along which the support is configured to fold in use. For example the prefolds may be formed by folding the material during manufacture or by scoring or perforation of the material. In some embodiment a mixture of prefold methods may be used to provide the desired sequence of folding when a user erects the support from its closed to open position . . . .

Furthermore, although embodiments of the present invention have been described with reference to a base, it is to be understood that the outer support may have an open base which is closed by only the liner.

It will also be appreciated that the particular shape of the support in the open position will depend upon the particular shape and configuration of the support and the number and arrangement of the prefolds provided on the support. Thus, it will be appreciated that the skilled person may provide various arrangements which utilise features in accordance with one or more of the above embodiments of the invention.

The invention is not intended to be restricted to the details of the above-described embodiments. For instance, the sample collector can be used to collect other samples for example a stool sample or vomit.

1-31. (canceled)

32. A sample collector comprising:
an inner flexible collection bag having an opening along its upper edge at least when open for use; and
an outer support for supporting the collection bag at its opening, the support having sides that can fold together for storage and open to provide a structure in which one end has a spout and another end has a transverse portion, the sides including fold lines to enable bending of the sides from a storage configuration into an open configuration,
the collection bag being connected to the outer support such that on opening of the outer support the bag is held open for collection of a sample.

33. A collector as claimed in claim 32, in which the collection bag comprises a removable extension extending above the opening of the collection bag.

34. A collector as claimed in claim 32, in which in the outer support forms a prism configuration having a substantially triangular or wedge shaped cross-section in the open position.

35. A collector as claimed in claim 32, in which in the open position the opening has at least three sides.

36. A collector as claimed in claim 35, in which in the open position the transverse portion is substantially square or rectangular in shape.

37. A collector as claimed in claim 32, in which the outer support is substantially flat in the storage position.

38. A collector as claimed in claim 32, in which the sides are scored, perforated or pre-folded along fold lines to enable the support to be movable from the storage position to the open position.

39. A collector as claimed in claim 38, in which the support comprises a first end and a second opposed end, in which the second opposed end provides an opening for supporting the collection bag, and in which the support comprises at least one pair of perforations, score or pre-folds extending from the first end towards the second end.

40. A collector as claimed in claim 39, in which the at least one pair of perforations, scores or pre-folds diverge away from each other in a directions extending from the first end towards the second end.

41. A collector as claimed in claim 40, in which the at least one pair of perforations, scores or pre-folds converge towards each other in a directions extending from the first end towards the second end.

42. A collector as claimed in claim 32, in which the outer support further comprises at least one window cut out, and in which one or more of the window cut out(s) comprises at least one mark to indicate one or more of a minimum fill level and/or a maximum fill level.

43. A collector as claimed in claim 42, in which one or more window cut out comprises a temperature gauge.

44. A collector as claimed in claim 32, further comprising at least one test stick for insertion into the sample.

45. A collector as claimed in claim 44, in which one or more test stick is adhered to the inside of the collection bag, and in which the one or more test stick(s) adhered to the inside of the bag are sealed within the bag prior to use.

46. A collector as claimed in claim 45, in which one or more test sticks are provided on an extension of the support such that the test stick(s) are foldable into the sample.

47. A collector as claimed in claim 44, in which the outer support comprises at least one window cut out positioned for reading the results of the test stick.

48. A collector as claimed in claim 32, in which prior to use in the stored position, the collector bag is sealed along all of its edges, and in which the collector bag comprises micro-perforations or a laser tear line located at substantially the level of an opening provided by the outer support.

49. A collector as claimed in claim 32, in which the inner flexible collection bag comprises a seal located above the base of the collection bag to provide a false bottom located between the base of the bag and the seal, in which an opening is provided at the lowest point of the seal to allow a sample to pass through the seal into the false bottom.

50. A collector as claimed in claim 32, in which the outer support further comprises an extension for supporting a hypodermic needle, and in which the collection bag is arranged in use to be connected via a tube to the needle.

51. A kit for collecting a sample, the kit comprising method of collecting a liquid sample comprising:
a collector as claimed in claim 32; and
a sealable container for receiving a sample collected in the collector

52. A sample collector comprising:
an inner flexible collection bag having an opening along its upper edge at least when open for use; and
an outer support for supporting the collection bag at its opening, the support being formed with sides having fold lines configured to allow it to conform to:
a first shape in which the sides are substantially flat against one another; and
a second shape in which the outer support forms a prism-like configuration;
the collection bag being connected to the outer support such that on opening of the outer support the bag is held open for collection of a sample.

53. A sample collector comprising:
an inner flexible collection bag having an opening along its upper edge at least when open for use; and
an outer support for supporting the collection bag at its opening, the support having sides that can fold together between a storage configuration and an open configuration, the sides including fold lines to enable the outer support to be snapped from one position to the other and to remain stable and self-supporting in the open position;
the collection bag being connected to the outer support such that on opening of the outer support the bag is held open for collection of a sample.
